(19) United States
(12) Patent Application Publication
(10) Pub. No.: US 2005/0251041 A1
(43) Pub. Date: Nov. 10, 2005

(54) Doppler ultrasound processing system and method for concurrent acquisition of ultrasound signals at multiple carrier frequencies, embolus characterization system and method, and ultrasound transducer

(76) Inventor: Mark A. Moehring, Seattle, WA (US)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(21) Appl. No.: 10/840,940

(22) Filed: May 7, 2004

(51) Int. Cl. A61B 8/00; A61B 8/14; A61B 8/12

(52) U.S. Cl. 600/441; 600/437; 600/453; 600/455

(57) ABSTRACT

A Doppler ultrasound signal processing system for processing reflected ultrasound signals detected by an ultrasound transducer for multiple carrier frequencies concurrently. Additionally, an embolus characterization system and method, and an ultrasound transducer are included as well.

### STATEMENT AS TO GOVERNMENT RIGHTS

This invention includes embodiments that were made with United States Government support under Grant No. 1 R43 HL 64486-01A1 awarded by National Institutes of Health (NIH). The United States Government has certain rights in the invention.

### TECHNICAL FIELD

The present invention is related generally to ultrasound systems, and more particularly, to a Doppler ultrasound system and method, embolus characterization system and method, and ultrasound transducer that can be used for characterization of microembolic events.

### BACKGROUND OF THE INVENTION

An embolus is defined as a plug of clot, fat, air or other material, brought by blood flow from one location in the circulation to another, and obstructing flow in the vessel where it comes to rest. A microembolus is an embolus small enough so as not to block flow in major arteries, but large enough to obstruct flow at least temporarily in the terminal branches of arteries and/or the microcirculation. Embolism is associated with a large number of cardiovascular abnormalities including stroke, heart attacks, peripheral arterial diseases, venous thrombosis and pulmonary embolism. Doppler detected microemboli have also been associated with these conditions as well as procedures such as cardiovascular surgery and catheterizations. Microemboli have been implicated as the source of a variety of cerebral abnormalities, including the neurological deficit that often follows open heart surgery, and incidence of stroke and transient ischemic attacks associated with prosthetic valves. Microemboli detected by Doppler ultrasound during carotid artery surgery have been associated with MRI and CT detected brain lesions present following surgery.

Characterizing a microembolus as gaseous or non-gaseous (solid or liquid) is essential for clinical management of patients when the source of the microemboli is ambiguous—i.e., underlying pathology is not sufficiently known to determine the threat posed by the microembolus, and therefore the appropriate response. For example, middle cerebral artery (MCA) microemboli in a patient with ipsilateral carotid stenosis is >80% positive predictive of plaque ulceration and indicates the need for prompt surgical intervention. However, if the same patient has a mechanical heart valve, understanding that the emboli are all gaseous would dictate a less urgent and more conservative course of patient management.

Specific knowledge of embolic composition is anticipated to affect patient management in two ways. First, surgical techniques can be altered based on understanding which maneuvers produce microemboli of a given composition. For example, cross-clamping and cannulation are not restricted to one position on the aorta, and the cannula can be inserted at different positions within the aorta. Second, better therapeutic decisions can be made for pharmacological protection of the brain. Neuro-protective agents are the focus of recent research efforts and there is indication that they need to be introduced as closely as possible to the time of the insult to the brain for maximum effectiveness. Ultrasound differentiation of microembolus composition is projected to be a usefuil tool in determining when and which neuro-protective agents are appropriate.

Surgery Involving Cardiopulmonary Bypass

Substantial clinical significance of embolus characterization is found in the arena of surgery involving cardiopulmonary bypass (CPB). Worldwide, more then one million surgeries utilizing CPB are performed each year and 576,000 coronary artery bypass graft surgeries were performed in the US in 2001. The overall rate of stroke associated with CPB is conservatively 3-5%. Acute care for stroke victims who had CPB in association with coronary artery bypass grafting is alone between $0.6 and $1 billion per year in the U.S.; valve replacement patients consume an additional $225 to $315 million. The incidence of cerebral complications after cardiac surgery is related to increased age. The rate of stroke is <1% in coronary artery bypass graft (CABG) patients below 65 year of age, 5% in patients aged 65 or more and 7-9% in patients aged 75 or more. A correlation between intra-operative cerebral microembolic load and postoperative neuropsychological dysfunction has been demonstrated. Reported incidence of short-term cognitive decline varies widely (from 33-83%). A recent report from Duke Medical Center showed a decline in cognitive function in 42% of patients, five years after CABG surgery. A study in the Netherlands comparing two groups of patients undergoing coronary artery bypass grafting with and without cardiopulmonary bypass pump observed cognitive decline at 12 months in 31% of the off-pump group and in 34% of the on-pump group.

Approximately one third of strokes associated with CPB are intra-operative, and embolic strokes account for 62% of strokes. The sources of microemboli during CPB are numerous: perfusionist interventions at the pump, placement of the aortic cannulation, decannulation, cross-clamping of the aorta, cross-clamp release, and surgical entrainment of gas into the heart during open heart procedures.

Cerebral arterial gas embolism involves the migration of gas to small arteries (diameter ˜30-60 μm). The emboli cause changes by two mechanisms: a reduction in perfusion distal to the obstruction and an inflammatory response to the bubble, along with breakdown of the blood-brain barrier. Microbubbles are rapidly absorbed and therefore only briefly interrupt cerebral arteriolar flow, whereas large air emboli persist in the circulation for hours, causing primary ischemic injury with diffuse brain edema and increased intracranial pressure.

Based on autopsy material, patients who undergo CABG surgery are estimated to have as many as 15 million microemboli varying in size from 15-70 μm , trapped in cerebral arterioles. The clinical consequences of these microemboli is dependent not only on number but also on the composition (air bubbles, fat particles, platelet aggregates) and size of the emboli entering the microvasculature of the brain. Whether microemboli detected in the middle cerebral artery are from air bubbles introduced in the venous reservoir or from friable plaque shed from the ascending aorta is not always clear, and ultrasound differentiation would be useful throughout the surgery in eliminating ambiguities such as this and modifying surgical techniques. The effects of embolization are currently not appreciated until the patient comes out of anesthesia and manifests neurologic deficits in the intensive care unit, which is typically too late to take preventive or therapeutic action.

Mechanical Heart Valves

There were 82,000 prosthetic heart valves were implanted in 2001. Embolic stroke is a major complication in patients with mechanical heart valves, both during and after the operative period. The stroke rate for valve patients is generally higher than the 3-5% overall for procedures involving CPB quoted above. Post-surgically, somewhere between 50 and 90% of prosthetic heart valve recipients demonstrate microemboli in the middle cerebral artery, and the underlying composition is not completely understood. Recent work has shown that patients with mechanical heart valves who inhale 100% O2 demonstrate a ˜60% drop in the rate of microemboli detected in the MCA, implicating nitrogenous cavitation bubbles formed at the valve as one composition. Supporting this hypothesis is the significant increase in microembolic signals on exposure to a hyperbaric chamber. Non-gaseous emboli are by no means ruled out, especially in light of the fact that patients with prosthetic valves have much elevated stroke risk and incidence of transient ischemic attacks. Microemboli from heart valves do affect cognitive ability in the long term, but the underlying composition that yields this effect is not clearly delineated. Efforts to either protect the brain and/or eliminate the microemboli will benefit from differentiation of embolic composition. The composition of emboli has substantial therapeutic implications, for example, anticoagulation is not prescribed if air embolism is suspected.

Carotid Artery Surgery

Carotid endarterectomy (CEA) surgery is a widely used procedure and 128,000 were performed in the United States in 2001. Prevalence of stroke in this procedure is at best about 3% and is higher in many institutions. Intra-operative embolization has been estimated as the cause of perioperative stroke in up to 80% of CEA. Transcranial Doppler monitoring of the MCA during CEA enables detection of emboli and concurrent hemodynamic data on the adequacy of cerebral blood flow. Stroke resulting from intra-operative embolization depends on numerous factors, the most important being whether the emboli are surgically entrained gas bubbles or particulates shed from the surgical site.

Orthopedic Surgery

Forty percent of patients undergoing total hip replacement demonstrate cerebral microemboli. This is partly due to presence of patent foramen ovale (PFO), which is a communication between the right and left heart which allows venous blood containing microemboli of various compositions to enter the arterial side without going through the lungs. PFO has from 20 to 34% prevalence in the general population. Fat, which enters the circulation during high pressures involved in preparing the femur for prosthesis insertion, is understood to move through the lungs and into the arterial circulation in addition to transiting across PFOs. Air also has opportunity to enter the venous system during orthopedic surgery and during the fracture/trauma to bone/vasculature that results in orthopedic surgical intervention. Total knee arthroplasty is usually performed under tourniquet induced ischemia. Embolism to the pulmonary vasculature occurs frequently following deflation of tourniquets. Some patients develop fat embolism syndrome with respiratory compromise and neurological abnormalities in up to 80% of patients, ranging from mild disorientation to stupor and coma. The presence of microemboli in the cerebral arteries during orthopedic procedures leads again to the composition ambiguity that can be resolved by the ultrasound technique of this proposal. The proposed technology will be of use in orthopedic surgery when prevention or therapeutic methods to deal with various embolization constituents are introduced into practice.

Doppler ultrasound characterization of composition of microemboli would be of significant utility in both diagnosis and monitoring of microemboli and therapies to control them, and in further understanding of stroke mechanisms.

### SUMMARY OF THE INVENTION

The present invention includes a Doppler ultrasound signal processing system is provided for processing reflected ultrasound signals detected by an ultrasound transducer for multiple carrier frequencies concurrently. Additionally, an embolus characterization system and method, and an ultrasound transducer are included.

### BRIEF DESCRIPTION OF THE DRAWINGS

FIG. 1 is a partial block diagram of a Doppler ultrasound system according to an embodiment of the present invention.

FIG. 2 illustrates a process for generating Doppler shift data according to an embodiment of the present invention.

FIG. 3 illustrates a sub-process of the process shown in FIG. 2 for generating Doppler shift coordinates according to an embodiment of the present invention.

FIG. 4 is a partial block diagram of a Doppler ultrasound system according to another embodiment of the present invention.

FIG. 5 is a partial block diagram of a Doppler ultrasound system according to another embodiment of the present invention.

FIG. 6 is a flow diagram for an embolus characterization algorithm according to an embodiment of the present invention that can be used with a Doppler ultrasound system according to another embodiment of the present invention.

FIG. 7 is a diagram of an ultrasound transducer according to an embodiment of the present invention that can be used with a Doppler ultrasound system according to another embodiment of the present invention.

FIG. 8 is a diagram of ultrasound field patterns for an embodiment of the ultrasound transducer of FIG. 7.

FIG. 9 is a diagram of ultrasound field patterns for an embodiment of the ultrasound transducer of FIG. 7.

FIG. 10 is a diagram of an ultrasound transducer according to an embodiment of the present invention that can be used with a Doppler ultrasound system according to another embodiment of the present invention.

FIG. 11 is a diagram of ultrasound field patterns for an embodiment of the ultrasound transducer of FIG. 10.

### DETAILED DESCRIPTION OF PREFERRED EMBODIMENTS

The present invention includes embodiments of a Doppler ultrasound system capable of concurrent acquisition of reflected ultrasound signals at different carrier frequencies. Additionally, concurrent processing of the detected ultrasound signals at the different center frequencies to provide Doppler shift data may be provided by embodiments of the present invention. The processed data can be utilized for characterizing microemboli, such as distinguishing gaseous from non-gaseous microemboli. Methods and systems for characterizing emboli is provided by another aspect of the present invention. Additionally, ultrasound transducers and methods for detecting reflected ultrasound signals are provided by another aspect of the present invention. Certain details are set forth below to provide a sufficient understanding of the invention. However, it will be clear to one skilled in the art that the invention may be practiced without these particular details. In other instances, well-known circuits, control signals, and timing protocols have not been shown in detail in order to avoid unnecessarily obscuring the invention.

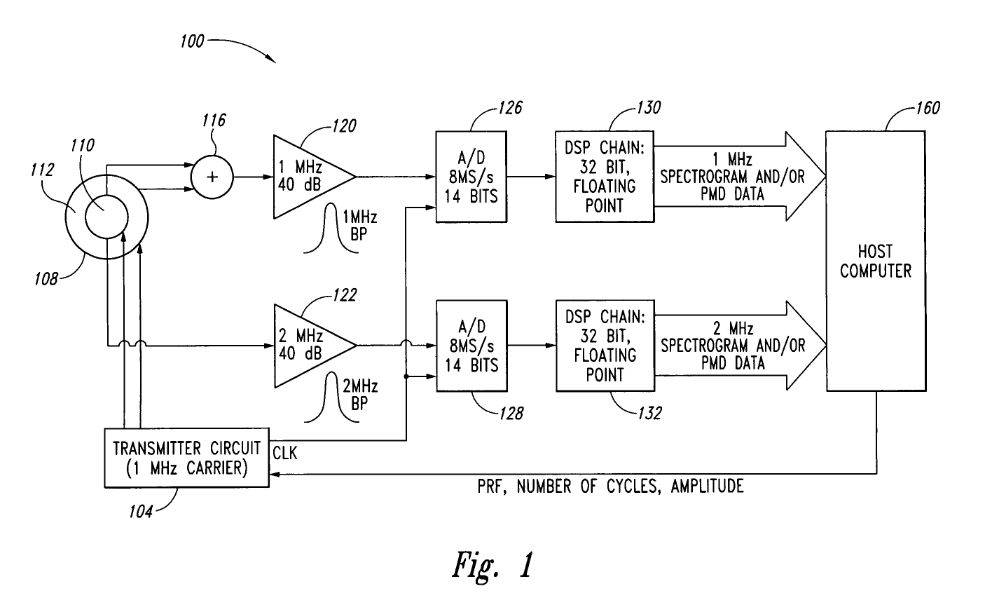

FIG. 1 illustrates portions of a Doppler ultrasound system 100 according to an embodiment of the present invention that is coupled to a host computer 160. The Doppler ultrasound system 100 includes a transmitter circuit 104 coupled to a transducer 108. The transducer 108 includes transducer elements 110, 112. The transducer element 110 represents a “piston” transducer element and the transducer element 112 represents an “annulus” transducer element. The transmitter circuit 104 drives the transducer elements 110, 112 to deliver an ultrasound signal at a selected carrier frequency. An example of a transducer having the arrangement of the transducer 108 is described in more detail below. The particular arrangement of the transducer 108 shown in FIG. 1 is not intended to limit the scope of the present invention. Alternative transducer arrangements can be utilized as well and remain within the scope of the present invention. It will be appreciated by those ordinarily skilled in the art that alternative transducer arrangements that can transmit ultrasound signals at a first frequency, and detect reflected ultrasound signals at the first frequency, and additionally at a second frequency, can be used with embodiments of the present invention, including single element transducers as well as multiple element transducers.

The transducer elements 110, 112 detect reflected ultrasound signals which are coupled to bandpass amplifier circuits 120, 122. The bandpass amplifier circuit 120 is a 1 MHz bandpass amplifier having a 150 kHz bandwidth and a 40 dB gain and the bandpass amplifier circuit 122 is a 2 MHz bandpass amplifier having a 300 kHz bandwidth and a 40 dB gain. An input of the bandpass amplifier circuit 120 is coupled to receive the output of a summing circuit 116 that sums the reflected ultrasound signals detected by both the transducer elements 110 and 112. In contrast, an input of the bandpass amplifier circuit 122 is coupled to receive the reflected ultrasound signals detected from only the transducer element 110. Information of the detected echo signals having a 1 MHz carrier and a 2 MHz carrier can be acquired concurrently.

One ordinarily skilled in the art will appreciate that high dynamic range analog-to-digital converters may be used such that the bandpass filtering discussed here can be done in the digital domain rather than having two analog pathways which are filtered and then digitized. Therefore the transition from analog to the digital domain, while preferred in this embodiment to be after bandpass filtering for each of the carrier frequencies of interest, is not predicated to be done in this fashion.

Coupled to outputs of the bandpass amplifier circuits 120, 122 are analog-to-digital converters (ADCs) 126, 128, respectively, to digitize the output signals of the respective bandpass amplifier circuits 120, 122. That is, the “raw echoes” detected by the transducer 108 and pre-amplified by the bandpass amplifier circuit 120, 122 are digitized to generate digital data representative of the raw echoes. Both the ADCs 126, 128, as well as the transmitter circuit 104, are clocked by a clock signal CLOCK so that the sampling rates of the ADCs 126, 128 are the same, and are synchronized with the start of each pulse period in order to prevent jitter in the resulting Doppler shift signal. As shown in FIG. 1, the ADCs 126, 128 both have 14-bit sample resolution and digitize the respective bandpass amplifier circuit output signal at an 8 MHz sample rate, or at 8 Msamples/second. Digital signal processing (DSP) circuits 130, 132 are coupled to a respective one of the ADCs 126, 128 to process the digitized output data and extract Doppler shift signal data relative to the respective channel carrier frequency. The digitized output data from the ADCs 126, 128 are processed by the DSP circuits 130, 132 to provide data representing Doppler blood flow velocity as well as Doppler power for multiple gate depths. The Doppler blood flow velocity can include data representing various velocity values, such as mean velocity, maximum velocity at each gate depth, and the like. Additionally, spectrogram data for at least one gate depth and at each carrier frequency can be provided. In an alternative embodiment, spectrogram data for at least one gate depth and for each carrier frequency is provided alone.

A host computer is coupled to the Doppler ultrasound system 100. The processed data is preferably provided by the Doppler ultrasound system 100 to the host computer 160 in the form of a power M-mode Doppler (PMD) image for display. With the processed data provided, the host computer 160 can display a PMD image for the 1 MHz channel, a PMD image for the 2 MHz channel, and if the data is provided, spectrograms for the 1 MHz and 2 MHz channels at respective gate depths. Additionally, the host computer 160 provides data to the transmitter circuit 104 to select the transmitted carrier frequency, number of cycles per burst, pulse repetition frequency, and signal amplitude of the transmitted ultrasound signal. In response, the transmitter circuit 104 drives the transducer 108 accordingly. In one embodiment of the present invention, the transmitter circuit 104 drives the transducer 108 to transmit an ultrasound signal having a 12 cycle pulse at a 1 MHz carrier frequency, an 8 kHz pulse repetition frequency, and having a derated spatial peak temporal average intensity of ˜230 mW/cm². Higher intensity levels up to the FDA diagnostic limit of 720 mW/cm2 for peripheral vascular work can be realized with appropriate transmitter settings and with appropriate transducer composition. However, it will be appreciated by those ordinarily skilled in the art that alternative ultrasound signals can be used without departing from the scope of the present invention.

As previously discussed, the DSP circuits 130, 132 process the digitized output data from the ADCs 126, 128 to provide Doppler shift data for various depths along an ultrasound beam axis. An example of digital signal processing circuitry and methods are described in greater detail to U.S. Pat. No. 6,196,972 to Moehring, issued Mar. 6, 2001, which is incorporated herein by reference. However, alternative circuits and methods can be utilized as well without departing from the scope of the present invention. It will be appreciated that those ordinarily skilled in the art will obtain sufficient understanding from the description provided herein to practice embodiments of the present invention.

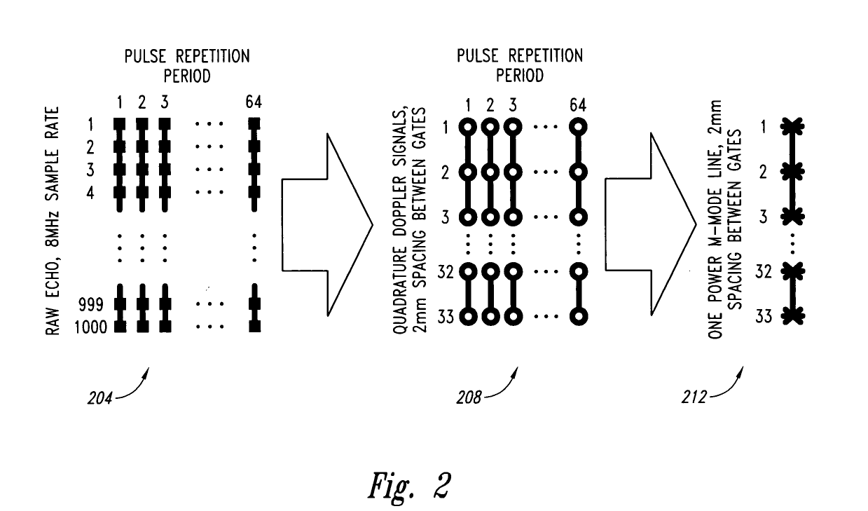

FIG. 2 illustrates a processing step of the DSP circuits 130, 132 according to an embodiment of the present invention. As shown in FIG. 2, the data representing the raw echoes 204 from the depth range of interest are demodulated, decimated and low pass filtered into Doppler (I,Q) pairs 208 that stratify the depth range of interest along the Doppler beam. A suitable method for decimation and low pass filtering are described in greater detail in the aforementioned U.S. Pat. No. 6,196,972. However, it will be appreciated that other methods can be used without departing from the scope of the present invention. The depth range of interest for an embodiment of the present invention is 22-86 mm to bracket the middle cerebral artery territory, as viewed from the temporal window, and sampled for the range in 2 mm increments. As a result, blood velocity and associated power is calculated for a complement of 33 range gates is provided.

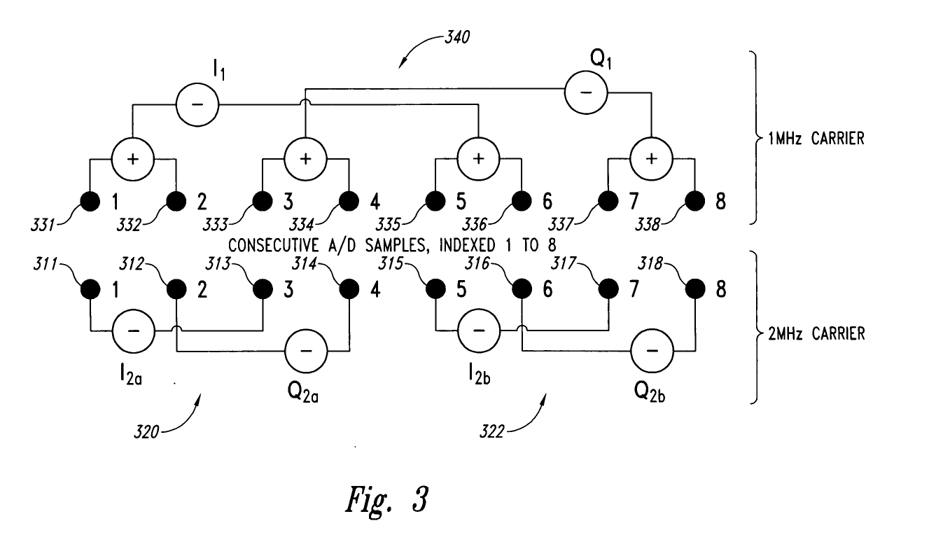

FIG. 3 illustrates in greater detail the demodulation of the data representing the raw echoes 204 (FIG. 2) where the digital demodulation technique for the 1 MHz and 2 MHz carriers using an 8 MHz sampling rate. For the 2 MHz carrier, the successive points 311-318 at the 8 MHz sampling rate correspond to carrier phase changes of 90°. Therefore, the difference between the first and third samples in any group of 4 successive samples (e.g., points 311 and 313 for the points 311-314; and points 315 and 317 for the points 315-318) is a measure of Doppler shift along an “in-phase” or “real” axis of the complex plane where Doppler shift signals “live.” Additionally, the difference between the second and fourth samples (e.g., points 312 and 314 for the points 311-314; and points 316 and 318 for the points 315-318) is a companion measurement that represents a measure of Doppler shift at essentially the same time and position, but on the a “quadrature” or “imaginary” axis in the complex plane. In this fashion, the 8 successive points 311-318 will yield two Doppler shift coordinates (I2a,Q2a) 320 and (I2b,Q2b) 322 for the 2 MHz carrier. For the 1 MHz carrier, the successive points 331-338 from the 8 MHz sampling rate represent phase changes of 45°. Therefore, averaging can be employed to distill out the 90° separated samples from which to calculate the Doppler shift sample (I1,Q1) 340. For example, the difference between the averages of points 331, 332 and 335, 336 can be used as a measure of Doppler shift along the in-phase axis and the difference between the averages of points 333, 334 and 337, 338 can be used as a measure of Doppler shift along the quadrature axis. Doppler shift data is processed for each pulse period and at each gate depth. The Doppler shift data is bundled into a matrix of 33 rows for corresponding range gates and 64 columns for corresponding pulse periods, and used to determine blood flow characteristics (e.g., power and velocity) across the complement of range gates.

Referring back to FIG. 2, the bundle of Doppler shift data is processed into PMD data 212 representing Doppler velocity and representing Doppler power at each gate depth. Data representing spectrograms for at least one gate depth for the 1 MHz and 2 MHz carriers can be added to the PMD data 212, or in an alternative embodiment, be the only data processed instead of the PMD data 212. As previously discussed, the PMD data 212 can be provided to a host computer 160, stored in a storage device, such as a hard drive, and used to display an image for channel 1 PMD, an image for channel 2 PMD, an image for channel 1 spectrogram, and an image channel 2 spectrogram. Additionally, the PMD data 212 provided by the DSP circuits 130, 132 can be used for characterizing microemboli, as will be discussed in more detail below.

The previously described embodiments have been provided by way of example. Consequently, various modifications can be made without departing from the scope of the present invention. For example, the Doppler ultrasound system 100 was described with respect to transmitting a 1 MHz ultrasound signal and concurrently acquiring reflected ultrasound signals at carrier frequencies of 1 MHz and 2 MHz. However, the Doppler ultrasound system 100 can be modified to concurrently acquire reflected ultrasound signals at two different carrier frequencies. In one embodiment, the two different carrier frequencies are 1.1 MHz and 2.2 MHz. Choosing these two frequencies may be desirable where 1 MHz and 2 MHz carrier frequencies result in significant external AM-band radio interference. Additionally, reflected ultrasound signals from carrier frequencies which are not the second harmonic can be utilized as well, without departing from the scope of the present invention.

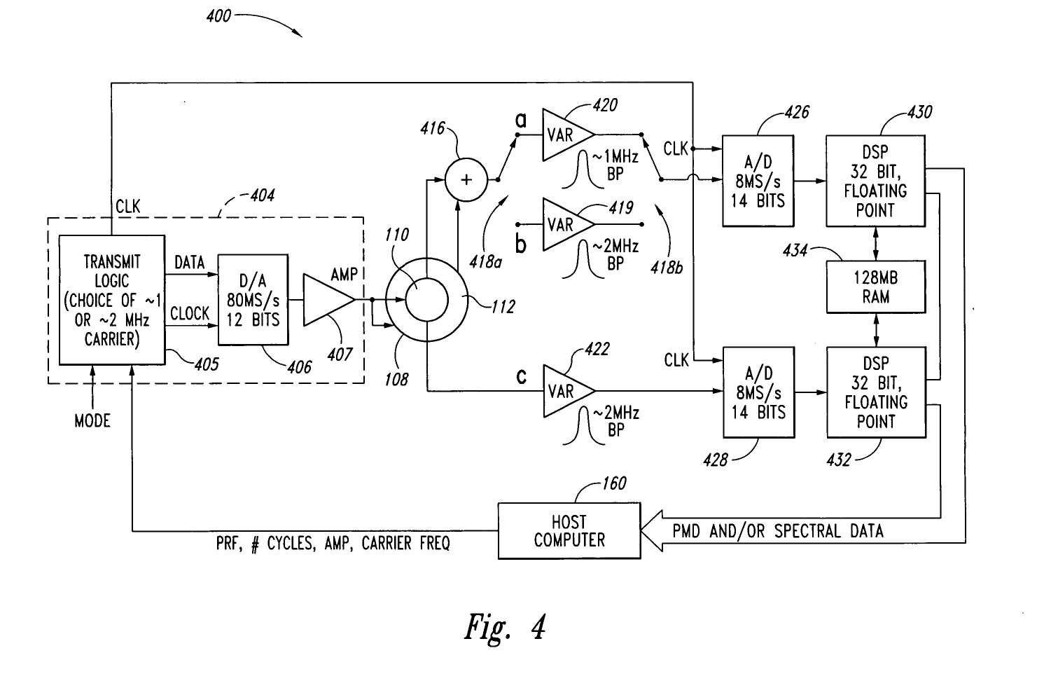

FIG. 4 illustrates portions of a Doppler ultrasound system 400 according to another embodiment of the present invention that is coupled to a host computer 160. As with the Doppler ultrasound system 100, the Doppler ultrasound system 400 is provided by way of example, and can be modified without departing from the scope of the present invention. The Doppler ultrasound system 400 is similar to the Doppler ultrasound system 100 shown in FIG. 1. However, the Doppler ultrasound system 400 includes an ultrasound transmitter and receiver for two modes of operation: (1) transmit at a first frequency, and receive at both the first frequency as well as a second frequency utilizing independent gain control and >70 dB channel separation between the first and second frequencies; and (2) transmit and receive at a third frequency for providing diagnostic ultrasound. The Doppler ultrasound system 400 is described with respect to a first frequency of 1 MHz, a second frequency of 2 MHz, and a third frequency that is twice the first frequency, that is, 2 MHz. However, alternative frequencies can be utilized without departing from the scope of the present invention.

The Doppler ultrasound system 400 includes a transmitter circuit 404 coupled to a dual-element transducer 108 having a first element 110 and a second element 112. The transmitter circuit 404 includes a transmit logic circuit 405, a digital-to-analog converter (DAC) 406, and an output amplifier 407 for driving the transducer 108 with a pulsing capability at 2 MHz and also at 1 MHz. Selection between the two frequencies is based on a mode signal MODE provided to the transmit logic circuit 405. The appropriate data and clock signals are generated by the transmit logic circuit and provided to the DAC 406, which in turn, provides an appropriate input signal to the output amplifier 407 to drive the transducer 108 accordingly. As shown in FIG. 4, the DAC 406 feeding the output amplifier 407 has a 12-bit resolution, or ˜72 dB of dynamic range and a high enough output rate that the outgoing burst will be well sampled and have minimum out of band energy.

The Doppler ultrasound system 400 includes three receiver pathways. Paths (a) and (b) share a summing circuit 416, analog-to-digital converter (ADC) 426 and digital signal processor (DSP) circuit 430. Path (a) includes a bandpass amplifier circuit 420 having a 1 MHz center frequency and path (b) includes a bandpass amplifier 419 having a 2 MHz center frequency. The switch 418a selectively couples the output of the summing circuit 416 to inputs of the bandpass amplifier circuits 420 or 419, and the switch 418b selectively couples the output of the bandpass amplifier circuits 420 or 419 to the input of the ADC 426. In this manner, paths (a) and (b) can be switched through switches 418a, 418b for utilizing the Doppler ultrasound system 400 for 2 MHz diagnostic work or 1 MHz embolus monitoring and characterization. Path (c) is generally represented by bandpass amplifier 422 having a center frequency of 2 MHz, ADC 428, and DSP circuit 432. Paths (a) and (c) provide processing of reflected ultrasound signals to provide PMD data and/or spectral data that can be used for characterization of microemboli.

An optional random access memory (RAM) 434 is coupled to the DSP circuits 430, 432 to provide a data buffer for storing commonly accessible data. With both DSP circuits 430, 432 having access to the RAM 434, processing of data stored therein can be shared between the two DSP circuits.

In operation, the receiver paths (a) and (b) process signals summed from the two transducer elements 110 and 112. Path (c), in contrast, processes signals from the center element 110 only, and does so for the anticipated second harmonic at 2 MHz carrier frequency. The DSPs 430, 432 are used to process data acquired from receiver paths (a) and (c) concurrently for embolus monitoring and characterization, or alternatively, one of the digital signal processors 430, 432 can be used to process data acquired from the receiver path (b) alone for 2 MHz diagnostic ultrasound.

The PMD and/or spectral data for the first and third frequencies are provided by the Doppler ultrasound system 400 to a host computer 160. As previously described with respect to the Doppler ultrasound system 100, the host computer can perform a variety of high level tasks including integrating both sets of PMD and/or spectral data in embolus monitoring/characterization mode, storing and displaying the PMD and/or spectral data, and replaying saved data. The host computer 160 can perform the high level tasks for the 2 MHz diagnostic mode PMD and/or spectral data as well. As shown in FIG. 4, the host computer 160 also provides data to the transmitter circuit 404 for the selection of transmitted carrier frequency, number of cycles per burst, pulse repetition frequency, and signal amplitude.

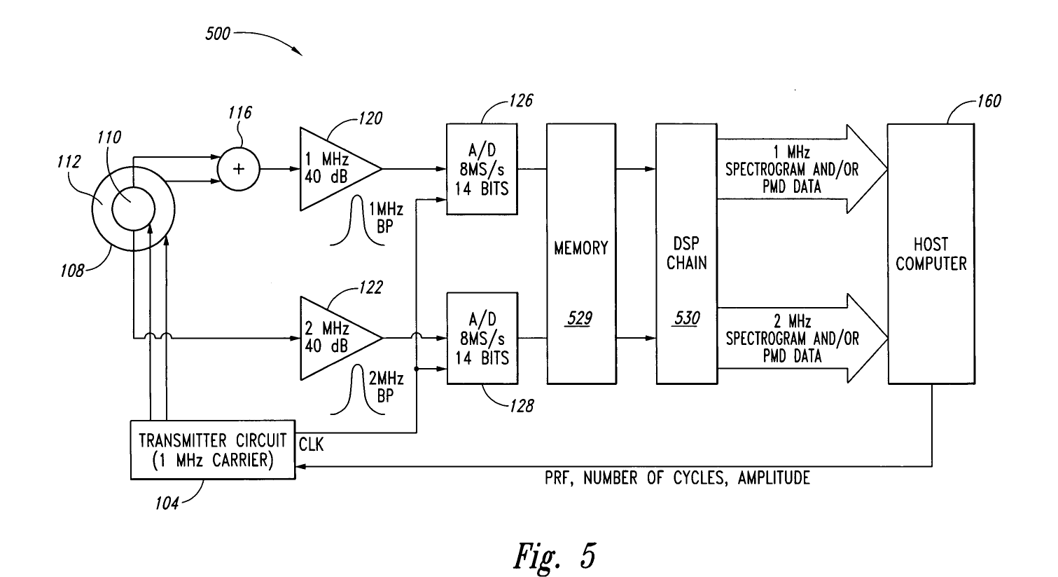

FIG. 5 illustrates portions of a Doppler ultrasound system 500 according to another embodiment of the present invention that is coupled to a host computer 160. The Doppler ultrasound systems 100 and 400 previously discussed included parallel processing capability for each frequency channel. One skilled in the art will appreciate that the processing speed of digital signal processors is growing dramatically year by year, and that the data acquired into the digital domain for the two carrier frequencies discussed in the preferred embodiment may be processed in parallel with two digital signal processing circuits, as with the Doppler ultrasound systems 100 and 400, or may be processed serially with one digital signal processing circuit, as illustrated in FIG. 5 for the Doppler ultrasound system 500. Thus, the scope of the present invention is not limited by whether the processing path is serial or parallel, or how many processors are utilized.

The Doppler ultrasound system 500 includes a transmitter circuit 104, a transducer 108, a summing circuit 116, bandpass amplifier circuits 120, 122, and analog-to-digital converters (ADCs) 126, 128 as previously described with respect to the Doppler ultrasound system 100. A conventional memory 529 is coupled to the ADCs 126, 128 to store the digitized data for processing by a digital signal processing (DSP) circuit 530. In contrast to the Doppler ultrasound system 100, which includes the DSP circuits 130, 132 for processing the digitized data in parallel, the Doppler ultrasound system uses the memory 529 to store the data output by the ADCs 126, 128 for processing by the DSP 530. Although processing of the data is not in parallel, acquisition of the digital data for the reflected ultrasound signals at the carrier frequencies of interest is concurrent. The DSP circuit 530 performs the Doppler processing as previously described with respect to the DSP circuits 130, 132 and 426, 428. However, processing of the digital data for the different carrier frequencies can be performed alternately or successively to provide the Doppler velocity information and the Doppler power information for the multiple gate depths for the different carrier frequencies. As shown in FIG. 5, two sets of Doppler shift information, one for each carrier frequency, is provided by the Doppler ultrasound system 500 to a host computer 160. As previously discussed with respect to the Doppler ultrasound systems 100 and 400, the host computer 160 can store and display the Doppler information. It will be appreciated that the Doppler ultrasound system 500 can be modified to include a diagnostic Doppler ultrasound channel, as previously described with respect to the Doppler ultrasound system 400.

As previously discussed, the PMD and/or spectral data provided by the Doppler ultrasound systems according to embodiments of the present invention can be used for characterizing microemboli. In a preferred embodiment, emboli are characterized as gaseous in composition if they exhibit a significant reflection at the fundamental carrier frequency (e.g., 1.1 MHz carrier frequency) and at the 2^{nd} harmonic carrier frequency (e.g., 2.2 MHz carrier frequency). Emboli are characterized as non-gaseous in composition if they exhibit a significant reflection at the fundamental carrier frequency and absence of reflection or signal level below a minimum reflection threshold reflection at the 2^{nd} harmonic carrier frequency. Methods for tracking and detecting these transient increases in energy are numerous and include delta follower methods and transient peak detection methods. For example, embolus detection methods are described in U.S. Pat. Nos. 6,524,249 and 6,547,736 to Moehring et al., issued Feb. 25, 2003 and Apr. 15, 2003, which are incorporated herein by reference.

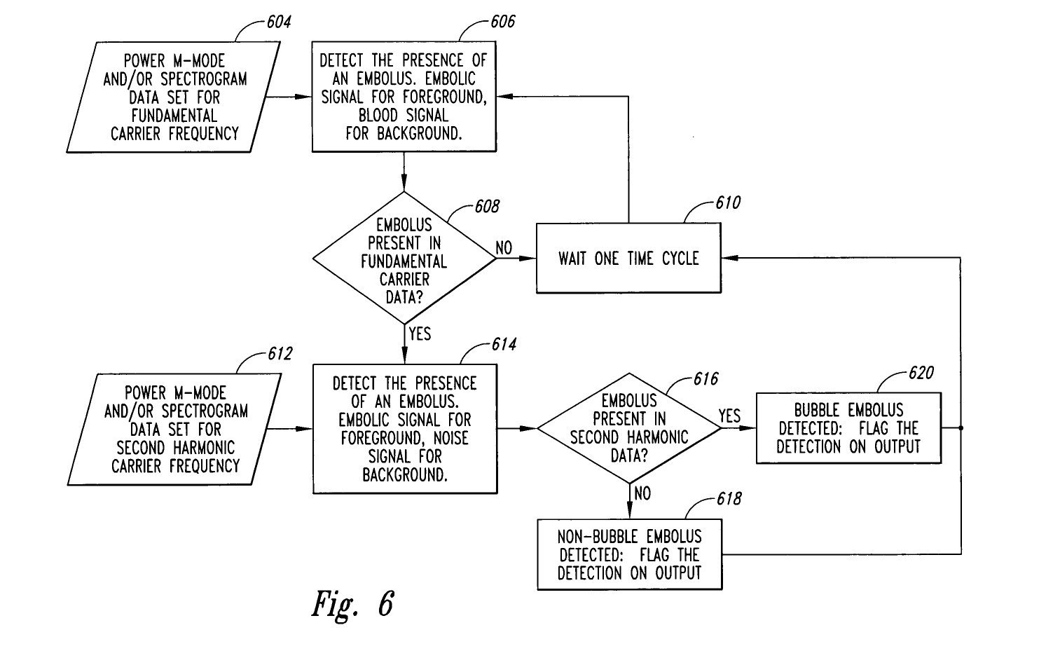

FIG. 6 illustrates a flow diagram illustrating a process 600 for characterizing emboli according to an embodiment of the present invention. In an embodiment of the present invention, the process 600 is a software algorithm performed by a host computer coupled to receive PMD and/or spectral data for a fundamental carrier frequency and for a second harmonic carrier frequency. In alternative embodiments, the process 600 can be performed by a combination of hardware processing circuits and software algorithm, portions of which can be included in a Doppler ultrasound system according to an embodiment of the present invention, or the host computer, or both. Additionally, as will be explained below in more detail, embodiments of a characterization process according to the present invention can be utilized with conventional Doppler ultrasound systems that can perform spectral analysis of reflected ultrasound signals at first and second carrier frequencies.

The primary inputs for the embolus characterization analysis algorithm are (a) the ratio of signal to background—EBR or “embolus to blood power ratio”—in dB, from the fundamental carrier frequency data, and (b) the ratio of signal to background—ENR or “embolus to noise ratio”—in dB, from the second harmonic data. The ENR concept is a variant of the “embolus to blood ratio”—EBR—which is described in greater detail with respect to detecting the presence of emboli in flowing blood in the aforementioned U.S. Pat. Nos. 6,524,249 and 6,547,736. In ENR and EBR, the “E” represents power from the embolic signal, “N” is power from noise, “B” is power from blood backscatter, and “R” stands for ratio. Backscatter from blood, which is fundamentally a linear process, does not appear in the second harmonic response. Similarly, backscatter from any non-gaseous entity such as blood clot, or fat globule, is also a linear process, and does not appear in the second harmonic response. The backscatter from bubbles is non-linear and does have a second harmonic component which is used in embodiments of the present invention to discriminate a gaseous embolus from a non-gaseous embolus.

There are different methods for calculating the EBR and the ENR, including (1) measuring the peak amplitude of the power M-mode Doppler data versus the background amplitude at the same gate depth, and (2) measuring the embolus signature peak amplitude in a spectrogram and comparing with the background amplitude at a different part of the spectrogram (i.e., a region with no signal). In the latter case, spectrogram processing can be done at each gate depth in the M-mode data so that emboli at all depths can be sensitively detected. The absence of signal should result in an ENR value of 0 dB (i.e., E=N), and this happens in the second harmonic signal (power M-mode data as well as spectrogram data). A localized neighborhood assessment of signal power can be used to assess signal energy, E.

As shown in FIG. 6, at steps 604, 606 embolus detection is first performed for the fundamental frequency data based on the Doppler power data and/or spectrogram data, such as that provided by a Doppler ultrasound system according to an embodiment of the present invention. At step 608, a determination is made based on the assessment of the energy behavior from the step 606. If the energy behavior is not indicative of an embolic event, the Doppler power data and/or spectrogram data for a next time cycle is evaluated at step 610. However, if the energy behavior is indicative of an embolic event, then the corresponding spatial-temporal region in the second harmonic data is reviewed for an embolic event at steps 612 and 614.

A calculation of signal power in a region where there clearly is no embolic presence is used to assess background energy level, N. As described in the aforementioned U.S. Pat. Nos. 6,524,249 and 6,547,736, the background can be assessed in the power M-mode Doppler domain by observing time periods when no embolus is present. An alternative approach for background assessment in the spectral domain is described in U.S. Pat. No. 5,348,015 to Moehring et al., issued Sep. 20, 1994, which is incorporated herein by reference. Constructs such as delta followers or simple averages (mean or median) over long periods of no embolic signal provide good background measurements of power, and are described in the aforementioned patents. In the second harmonic data, E and N are expected to be equal when no signal is present, resulting in an ENR of 0 dB. Since these measurements of E and N are done on different regions in the spectrogram, and there is an underlying noise process, the resulting ratio can produce a negative or positive dB value, but should be within an acceptable energy threshold from 0 dB.

At step 616, the determination is made whether an embolic event is present based on the assessment from the steps 612, 614. In the event that an embolic event is not detected at the step 616 (i.e., an embolic event was detected for the data set for the findamental carrier frequency, but not detected for the data set for the second harmonic carrier frequency), a flag indicating that a non-gaseous embolus was detected is output. However, in the event that an embolic event is detected at the step 620 (i.e., embolic events are detected for the data sets for both the fundamental carrier frequency and the second harmonic carrier frequency), a flag indicating that a gaseous embolus was detected is output. Following the steps 618 or 620, the process returns to the step 610 to evaluate the Doppler power data and/or spectrogram data for a next time cycle. The process of making the assessment based on the Doppler data set for the fundamental carrier frequency, and if necessary, the assessment based on the Doppler data set for the second harmonic carrier frequency, is repeated to continuously characterize embolic events as gaseous or non-gaseous. In this manner, the flags output by the process 600 can be accumulated and used to provide information on the characterization of embolic events to a user.

As previously discussed, a dual-element ultrasound transducer can be used with Doppler ultrasound systems according to an embodiment of the present invention. FIG. 7 illustrates a dual-element ultrasound transducer 700 according to an embodiment of the present invention. The dual-element transducer 700 includes a center “piston” transducer 702 and an outer annulus transducer 704. The ultrasound transducer 700 transmits and receives on both elements 702, 704 at a first carrier frequency, and receives on the center element 702 at a second carrier frequency. As will be explained in more detail below, the ultrasound transducer 700 is configured such that the beam pattern for the first carrier frequency and the beam patter for the second carrier frequency are similar. The term “beam pattern” as used herein is synonymous with, and used interchangeably with, alternative terms such as “spatial sensitivity pattern,” “sample volume,” “ultrasound field pattern,” and the like, as well known in the art.

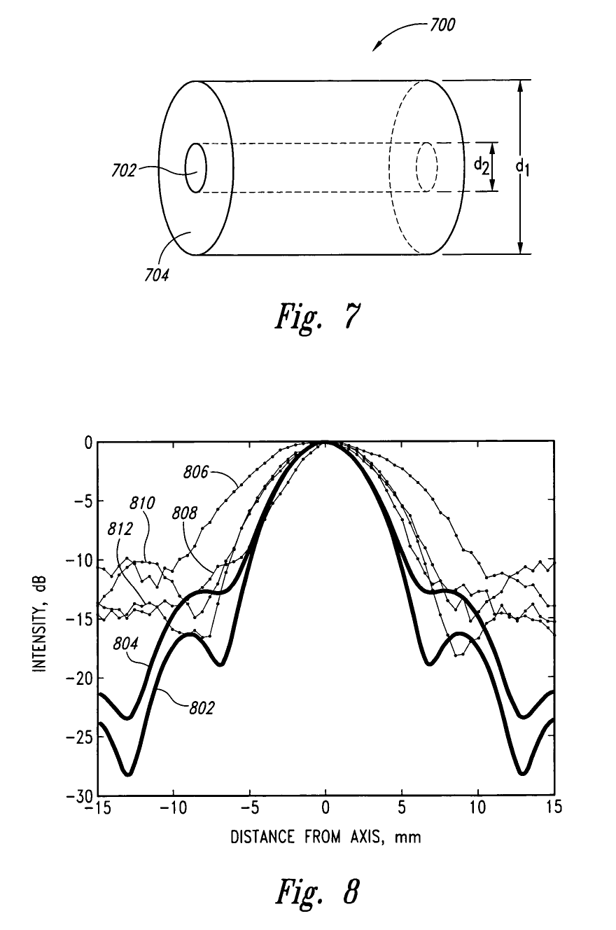

By having spatially similar sensitivity patterns at the first and second carrier frequencies, embolic events are “visible” at both carrier frequencies and from the same spatial region for each carrier frequency. A mismatch of the sample volumes at the two carrier frequencies may result in an embolus being observed at one frequency and not at the second frequency because of field pattern factors and not because of embolus composition factors. With specific reference to a first carrier frequency of 1 MHz and a second carrier frequency equal to the second harmonic of the first carrier frequency, that is, 2 MHz, as generally known, the width of the main lobe of the ultrasound sensitivity pattern is similar for 1 MHz and 2 MHz if the 1 MHz field is generated from a flat piston transducer (i.e., both the center piston transducer 702 and the outer annulus transducer 704) which has twice the diameter d₁ of the diameter d₂ of the 2 MHz transducer crystal (i.e., only the center piston transducer 702). Following this general design rule yielded a dual-element transducer having the sensitivity pattern for the 1 MHz and 2 MHz carrier frequencies as shown in FIG. 8.

As shown in FIG. 8, although there are differences in the first side lobe levels between the two different frequencies, the resulting dual-element transducer is acceptable where the territory of blood flow being interrogated is away from the first side lobe levels. Curves 802 and 804 represents the theoretical ultrasound field pattern at the 2 MHz and 1 MHz carrier frequencies, respectively. The theoretical profiles were obtained using an ultrasound field simulation software, FIELD II matlab routines by J. A. Jensen, assuming a 7 mm piston diameter, 14 mm annulus outer diameter, 1 MHz and 2 MHz carrier frequencies, 8 μs pulse length, and 8 kHz pulse repetition frequency. Curves 806 and 808 represent the measured ultrasound field pattern along the lateral axes for the 1 MHz carrier frequency and curves 810 and 812 represent the measured ultrasound field pattern along the lateral axes for the 2 MHz carrier frequency. Curves 806, 808, 810, and 812 are measured from a transducer with center frequency 1.5 MHz and broad bandwidth to include 1 MHz and 2 MHz as operable frequencies.

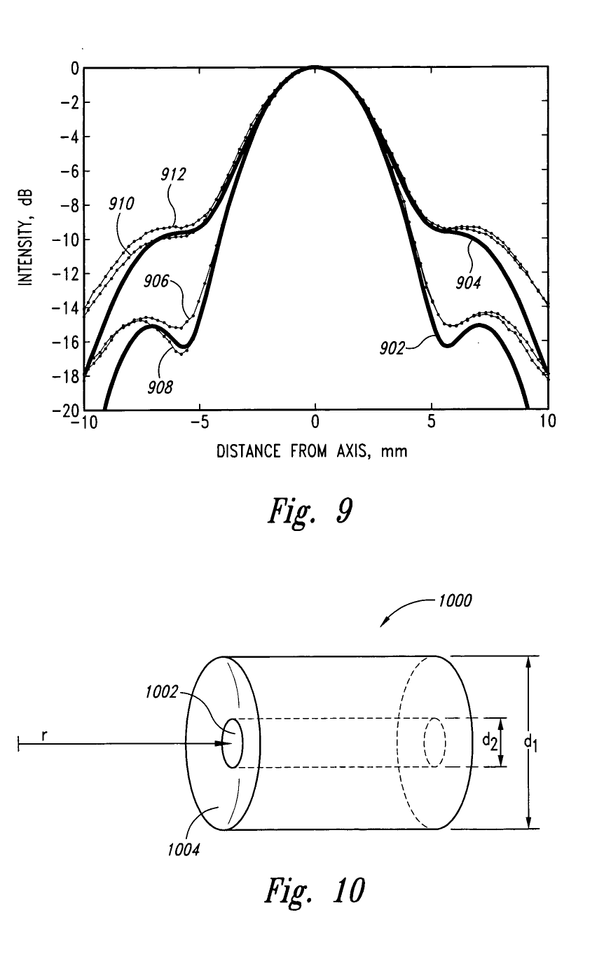

In an alternative embodiment, the dual-element ultrasound transducer 700 is constructed from one crystal having two resonance modes that could be varied by altering the construction parameters of the composite crystal. Parameters can be set so the resonances occurred at 1 MHz and 2 MHz. The sensitivity pattern for this embodiment is shown in FIG. 9. As shown in FIG. 9, the intensity sampling on orthogonal axes at each fixed frequency are very similar, indicating good circular symmetry in the probe sensitivity pattern. Additionally, with the embodiment of the dual-element ultrasound transducer 700, an intensity level of 230 mW/cm2 could easily be accomplished with peak voltage levels of ˜80 Vpp. This intensity is sufficient to penetrate skull (temporal bone) in most patients. Curves 902 and 904 represents the theoretical ultrasound field pattern at the 2 MHz and 1 MHz carrier frequencies, respectively. Curves 906 and 908 represent the measured ultrasound field pattern along the lateral axes for the 1 MHz carrier frequency and curves 910 and 912 represent the measured ultrasound field pattern along the lateral axes for the 2 MHz carrier frequency.

The main lobes of the ultrasound field pattern for carrier frequencies of 1.1 MHz and 2.2 MHz are well matched. The main lobe is fairly broad and has ˜5 mm full width at the −3 dB power point. This should be adequate for placing the highest sensitivity portion of the main lobe of the field pattern into the middle cerebral artery and then monitoring for microemboli. As shown in FIG. 9, the 1.1 MHz side-lobe is about 6-7 dB greater than that for 2.2 MHz. In scenarios such as when the distal internal carotid artery sits in the side lobe of the ultrasound field pattern while the proximal middle cerebral artery is in the main lobe, it may be desirable for the side lobes of the ultrasound field patterns at 1.1 MHz and 2.2 MHz to be more matched. That is, an embolus in the distal internal carotid which is a bubble may have a compromised second harmonic signature and a perfectly strong fundamental signature, and this runs the risk of misclassification as a non-gaseous embolus.

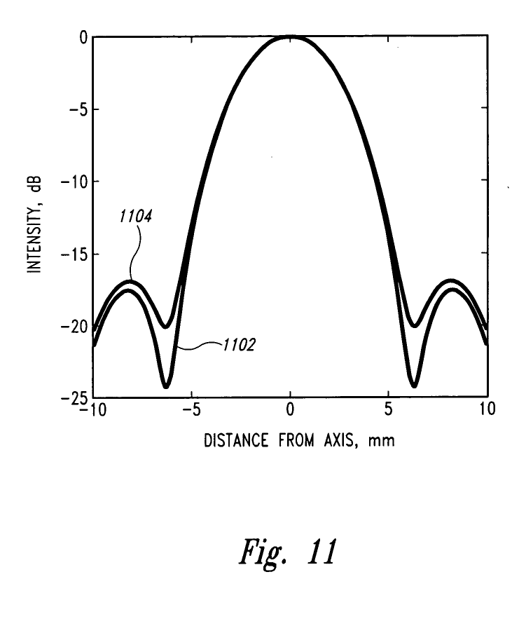

FIG. 10 illustrates a dual-element transducer 1000 according to alternative embodiment of the present invention. The dual-element transducer 1000 includes a center piston transducer 1002 and an outer annulus transducer 1004. The transducer 1000 has an overall outside diameter of d₁, and the piston transducer 1002 has an outside diameter d₂. As with the ultrasound transducer 700 (FIG. 7), the ultrasound transducer 1000 transmits and receives on both elements 1002, 1004 at a first carrier frequency, and receives on the center element 1002 at a second carrier frequency. The ultrasound transducer 1000 is configured such that the beam pattern for the first carrier frequency and the beam patter for the second carrier frequency are similar. However, in contrast to the transducer 700 shown in FIG. 7, the transducer 1000 includes a radius of curvature r. The simulated ultrasound field pattern for an 80 mm radius of curvature using an 8 μs pulse length, 14 mm annulus outer diameter, and 7 mm piston outer diameter, and 50 mm sample gate depth is shown in FIG. 11. Curves 1102 and 1104 represents the theoretical ultrasound field pattern at the 2.2 MHz and 1.1 MHz carrier frequencies, respectively. As shown in 11, the simulated ultrasound field patterns for 1.1 MHz and 2.2 MHz carrier frequencies demonstrate improved side lobe matching compared to the ultrasound field patterns shown in FIGS. 8 and 9.

It will be appreciated by those ordinarily skilled in the art that the embodiments of the transducers described with respect to FIGS. 7 and 10 can be modified without departing from the scope of the present invention. For example, the radius of curvature r can be changed from 80 mm, as well as the ratio between the outside diameter of the ultrasound transducer and the outside diameter of the piston element and remain within the scope of the present invention. Additionally, the similarity of the of the ultrasound field patterns between the first and second carrier frequencies may vary without departing from the scope of the present invention. As previously discussed, the desirable level of similarity may be in some part influenced by the particular application in which the ultrasound transducer is used. Different materials that are well known, as well as those later developed, can also be utilized in the transducer embodiments without departing from the scope of the present invention.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A Doppler ultrasound signal processing system for processing reflected ultrasound signals detected by an ultrasound transducer, the system comprising:
a first receiver circuit coupled to receive the reflected ultrasound signals detected by the ultrasound transducer and bandpass filter the same at a first carrier frequency;
a second receiver circuit coupled to receive the reflected ultrasound signals detected by the ultrasound transducer and bandpass filter the same at a second carrier frequency;
first and second analog-to-digital converter (ADC) circuits coupled to the first and second receiver circuits to concurrently generate first and second echo data for a plurality of depths, all respectively; and
a digital signal processing circuit coupled to the first and second ADC circuits to process the first echo data to generate first Doppler velocity data and first Doppler power data for the plurality of depths and process the second echo data to generate second Doppler velocity data and second Doppler power data for the plurality of depths.

2. The Doppler ultrasound signal processing system of claim 1 wherein each of the first and second receiver circuits comprises:
a bandpass filter amplifier having a center frequency at the respective carrier frequency to provide a respective output signal.

3. The Doppler ultrasound signal processing system of claim 1 wherein the digital signal processing circuit is adapted to calculate first Doppler mean velocity data for the plurality of depths from the first echo data and adapted to calculate second Doppler mean velocity data for the plurality of depths from the second echo data.

4. The Doppler ultrasound signal processing system of claim 1 wherein the digital signal processing circuit is adapted to calculate first Doppler maximum velocity data for at least some of the plurality of depths from the first echo data and adapted to calculate second Doppler maximum velocity data for at least some of the plurality of depths from the second echo data.

5. The Doppler ultrasound signal processing system of claim 1 wherein the digital signal processing circuit comprises:
a first Doppler processing path coupled to the first ADC circuit to process the first echo data to generate the first Doppler velocity data and the first Doppler power data for the plurality of depths; and
a second Doppler processing path coupled to the second ADC circuit to process the second echo data to generate the second Doppler velocity data and the second Doppler power data for the plurality of depths.

6. The Doppler ultrasound signal processing system of claim 5 wherein each of the first and second Doppler processing paths comprise:
a first processing stage to process the respective echo data and generate Doppler shift data for the plurality of depths; and
a second processing stage coupled to the first processing stage to process the Doppler shift data and generate the Doppler velocity data and the Doppler power data for the plurality of depths.

7. The Doppler ultrasound signal processing system of claim 1, further comprising:
a transmitting circuit coupled to the transducer to drive the transducer to transmit a diagnostic ultrasound signal having a carrier at a third carrier frequency;
a third receiver circuit coupled to receive the reflected ultrasound signals detected by the ultrasound transducer and bandpass filter the same at the third carrier frequency;
a first switch coupled to the ultrasound transducer and the first and third receiver circuits to selectively couple either the first or third receiver circuits to the ultrasound transducer; and
a second switch coupled to the first ADC circuit and the first and third receiver circuits to selectively couple either the first or third receiver circuit to the first ADC circuit.

8. The Doppler ultrasound signal processing system of claim 1, further comprising a memory circuit coupled to the first and second ADC circuits to store the first and second echo data for the plurality of depths, and the digital signal processing circuit comprises:
a first processing stage coupled to the memory circuit to access the first and second echo data stored therein and generate first Doppler shift data for the plurality of depths from the first echo data and generate second Doppler shift data for the plurality of depths from the second echo data; and
a second processing stage coupled to the first processing stage to process the first Doppler shift data to generate the first Doppler velocity data and the first Doppler power data for the plurality of depths and process the second Doppler shift data to generate the second Doppler velocity data and the second Doppler power data for the plurality of depths.

9. The Doppler ultrasound signal processing system of claim 1 wherein the second carrier frequency is a harmonic of the first carrier frequency.

10. The Doppler ultrasound signal processing system of claim 1 wherein the digital signal processing circuit is further configured to process the first echo data to generate first spectrogram data for a first depth of the plurality of depths and process the second echo data to generate second spectrogram data for a second depth of the plurality of depths.

11. A Doppler ultrasound system, comprising:
a transducer having first and second transducer elements;
a transmitter circuit coupled to the transducer to drive the first and second transducer elements to transmit a pulsed ultrasound signal having a carrier at a first carrier frequency;
a summing circuit coupled to the first and second transducer elements to output a sum signal representing the sum of the reflected ultrasound signals detected by the first and second transducer elements;
a first receiver circuit coupled to the summing circuit to bandpass filter the sum signal at a second carrier frequency and generate first raw echo data for a plurality of depths therefrom;
a second receiver circuit coupled to the first transducer element to bandpass filter the reflected ultrasound signals detected by the first transducer at a third carrier frequency and to generate second raw echo data for the plurality of depths therefrom; and
a digital signal processing circuit coupled to the first and second receiver circuits to process the first raw echo data to generate first Doppler velocity data and first Doppler power data for the plurality of depths and process the second raw echo data to generate second Doppler velocity data and second Doppler power data for the plurality of depths.

12. The Doppler ultrasound system of claim 11 wherein the first and second receivers comprise:
a bandpass filter amplifier having a center frequency at the respective carrier frequency to provide a respective output signal; and
an analog-to-digital converter coupled to an output of the bandpass filter amplifier to convert the respective output signal into digital data.

13. The Doppler ultrasound system of claim 11 wherein the digital signal processing circuit is adapted to calculate first Doppler mean velocity data for the plurality of depths from the first raw echo data and adapted to calculate second Doppler mean velocity data for the plurality of depths from the second raw echo data.

14. The Doppler ultrasound system of claim 11 wherein the digital signal processing circuit is adapted to calculate first Doppler maximum velocity data for at least some of the plurality of depths from the first raw echo data and adapted to calculate second Doppler maximum velocity data for at least some of the plurality of depths from the second raw echo data.

15. The Doppler ultrasound signal processing system of claim 11 wherein the digital signal processing circuit comprises:
a first Doppler processing path coupled to the first receiver circuit to process the first raw echo data and generate the first Doppler velocity data and the first Doppler power data for the plurality of depths; and
a second Doppler processing path coupled to the second receiver circuit to process the second raw echo data and generate the second Doppler velocity data and the second Doppler power data for the plurality of depths.

16. The Doppler ultrasound signal processing system of claim 15 wherein each of the first and second Doppler processing paths comprise:
a first processing stage to process the respective raw echo data and generate Doppler shift data for the plurality of depths; and
a second processing stage coupled to the first processing stage to process the Doppler shift data and generate the Doppler velocity data and the Doppler power data for the plurality of depths.

17. The Doppler ultrasound signal processing system of claim 11 wherein the transmitter circuit is further configured to drive the transducer to transmit a diagnostic ultrasound signal having a carrier at a fourth carrier frequency, and the Doppler ultrasound signal processing system further comprises:
a third receiver circuit coupled to receive the sum signal and bandpass filter the same for the fourth carrier frequency;
a first switch coupled to the summing circuit and the first and third receiver circuits to selectively couple either the first or third receiver circuits to the summing circuit; and
a second switch coupled to the digital signal processing circuit and the first and third receiver circuits to selectively couple either the first or third receiver circuit to the digital signal processing circuit.

18. The Doppler ultrasound signal processing system of claim 11, further comprising a memory circuit coupled to the first and second receiver circuits to store the first and second raw echo data for the plurality of depths, and wherein the digital signal processing circuit comprises:
a first processing stage coupled to the memory circuit to access the first and second raw echo data stored therein and generate first Doppler shift data for the plurality of depths from the first raw echo data and generate second Doppler shift data for the plurality of depths from the second raw echo data; and
a second processing stage coupled to the first processing stage to process the first Doppler shift data to generate the first Doppler velocity data and the first Doppler power data for the plurality of depths and process the second Doppler shift data to generate the second Doppler velocity data and the second Doppler power data for the plurality of depths.

19. The Doppler ultrasound signal processing system of claim 11 wherein the first and second carrier frequencies are the same frequency.

20. The Doppler ultrasound signal processing system of claim 19 wherein the third carrier frequency is a harmonic of the second carrier frequency.

21. The Doppler ultrasound signal processing system of claim 11 wherein the digital signal processing circuit is further configured to process the first echo data to generate first spectrogram data for a first depth of the plurality of depths and process the second echo data to generate second spectrogram data for a second depth of the plurality of depths.

22. A method for Doppler processing ultrasound signals detected by an ultrasound transducer, the method comprising:
generating first echo data for a first carrier frequency for a plurality of depths from the ultrasound signals detected by the ultrasound transducer;
concurrently generating second echo data for a second carrier frequency for the plurality of depths from the ultrasound signals detected by the ultrasound transducer; and
processing the first echo data to generate first Doppler velocity data and first Doppler power data for the plurality of depths and processing the second echo data to generate second Doppler velocity data and second Doppler power data for the plurality of depths.

23. The method of claim 22 wherein the second carrier frequency is a harmonic of the first carrier frequency.

24. The method of claim 22 wherein processing the first echo data to generate first Doppler velocity data comprises processing the first echo data to generate first Doppler mean velocity data for the plurality of depths from the first echo data and processing the second echo data to generate second Doppler mean velocity data for the plurality of depths from the second echo data.

25. The method of claim 22 wherein processing the first echo data to generate first Doppler velocity data comprises processing the first echo data to generate first Doppler maximum velocity data for at least some of the plurality of depths from the first echo data and processing the second echo data to generate second Doppler maximum velocity data for at least some of the plurality of depths from the second echo data.

26. The method of claim 22, further comprising processing the first echo data to generate first spectrogram data for a first depth of the plurality of depths and processing the second echo data to generate second spectrogram data for a second depth of the plurality of depths.

27. The method of claim 22 wherein generating the first echo data and generating the second echo data concurrently comprises:
bandpass filtering the ultrasound signals detected by the ultrasound transducer at a center frequency equal to respective carrier frequency; and
generating digital data representative of the bandpass filtered signals.

28. The method of claim 27 wherein bandpass filtering the ultrasound signals comprises digitally bandpass filtering the ultrasound signals.

29. The method of claim 22 wherein processing the first echo data and processing the second echo data comprises processing the first and second echo data in parallel through respective processing paths, the first processing path generating the first Doppler velocity data and the first Doppler power data and the second processing path generating the second Doppler velocity data and the second Doppler power data.

30. The method of claim 29 wherein processing the first and second echo data in parallel through the respective processing paths comprises:
generating first Doppler shift data for the plurality of depths from the first echo data;
generating second Doppler shift data for the plurality of depths from the second echo data;
processing the first Doppler shift data to generate the first Doppler velocity data and the first Doppler power data for the plurality of depths; and
processing the second Doppler shift data to generate the second Doppler velocity data and the second Doppler power data for the plurality of depths.

31. The method of claim 22 wherein processing the first echo data and processing the second echo data comprises:
storing the first and second echo data for the plurality of depths in a memory; and
accessing the first and second echo data stored in the memory;
generating first Doppler shift data for the plurality of depths from the first echo data;
generating second Doppler shift data for the plurality of depths from the second echo data;
processing the first Doppler shift data to generate the first Doppler velocity data and the first Doppler power data for the plurality of depths; and
processing the second Doppler shift data to generate the second Doppler velocity data and the second Doppler power data for the plurality of depths.

32. The method of claim 22 wherein the transducer comprises first and second transducer elements and generating first echo data comprises summing the ultrasound signals detected by the first and second transducers from which the first echo data is generated and generating second echo data comprises generating the second echo data from the ultrasound signals detected by the first transducer.

33. A method for processing ultrasound signals, comprising:
transmitting from a first ultrasound transducer a pulsed ultrasound signal having a carrier signal at a first carrier frequency;
transmitting from a second ultrasound transducer a pulsed ultrasound signal having a carrier signal at the first carrier frequency;
bandpass filtering reflected ultrasound signals detected by the first and second ultrasound transducer at a second carrier frequency;
generating first raw data from the bandpass filtered reflected ultrasound signals detected by the first and second ultrasound transducers for a plurality of depths;
bandpass filtering reflected ultrasound signals detected by the first ultrasound transducer at a third carrier frequency;
generating second raw data from the bandpass filtered reflected ultrasound signals detected by the first ultrasound transducer for the plurality of depths; and
processing the first raw data to generate first Doppler shift velocity data and first Doppler shift power data for the plurality of depths and process the second raw data to generate second Doppler shift velocity data and second Doppler shift power data for the plurality of depths.

34. The method of claim 33 wherein the first and second carrier frequencies are equal.

35. The method of claim 33 wherein processing the first raw data to generate first Doppler shift velocity data comprises processing the first raw data to generate first Doppler shift mean velocity data for the plurality of depths from the first raw data and processing the second raw data to generate second Doppler shift mean velocity data for the plurality of depths from the second raw data.

36. The method of claim 33 wherein processing the first raw data to generate first Doppler shift velocity data comprises processing the first raw data to generate first Doppler shift maximum velocity data for at least some of the plurality of depths from the first raw data and processing the second raw data to generate second Doppler shift maximum velocity data for at least some of the plurality of depths from the second raw data.

37. The method of claim 36 wherein the third carrier frequency is a harmonic of the second carrier frequency.

38. The method of claim 33, further comprising processing the first raw data to generate first spectrogram data for a first depth of the plurality of depths and processing the second raw data to generate second spectrogram data for a second depth of the plurality of depths.

39. The method of claim 33 wherein bandpass filtering the reflected ultrasound signals at the second carrier frequency and bandpass filtering the reflected ultrasound signals at the third carrier frequency comprises bandpass filtering the reflected ultrasound signals at a center frequency equal to respective carrier frequency, and generating the first raw data and generating the second raw data comprises generating digital data representative of the respective filtered signals for the plurality of depths.

40. The method of claim 39 wherein bandpass filtering the reflected ultrasound signals comprises digitally bandpass filtering the reflected ultrasound signals.

41. The method of claim 33 wherein processing the first raw data and processing the second raw data comprises processing the first and second raw data in parallel through respective processing paths, the first processing path generating the first Doppler shift velocity data and the first Doppler shift power data and the second processing path generating the second Doppler shift velocity data and the second Doppler shift power data.

42. The method of claim 41 wherein processing the first and second raw data in parallel through the respective processing paths comprises:
generating first Doppler shift data for the plurality of depths from the first raw data;
generating second Doppler shift data for the plurality of depths from the second raw data;
processing the first Doppler shift data to generate the first Doppler shift velocity data and the first Doppler shift power data for the plurality of depths; and
processing the second Doppler shift data to generate the second Doppler shift velocity data and the second Doppler shift power data for the plurality of depths.

43. The method of claim 33 wherein processing the first raw data and processing the second raw data comprises:
storing the first and second raw data for the plurality of depths in a memory; and
accessing the first and second raw data stored in the memory;
generating first Doppler shift data for the plurality of depths from the first raw data;
generating second Doppler shift data for the plurality of depths from the second raw data;
processing the first Doppler shift data to generate the first Doppler shift velocity data and the first Doppler shift data for the plurality of depths; and
processing the second Doppler shift data to generate the second Doppler shift velocity data and the second Doppler shift power data for the plurality of depths.

44. A method for characterizing emboli for a Doppler ultrasound system, comprising:
detecting the presence of an embolus based on power M-mode Doppler data representative of blood flow for a first carrier frequency;
determining whether an embolic signature corresponding to the detected embolus is present based on power M-mode Doppler data representative of the blood flow for a second carrier frequency; and
in response to determining that an embolic signature corresponding to the detected embolus is present, characterizing the detected embolus as gaseous, otherwise, characterizing the detected embolus as non-gaseous.

45. The method of claim 44 wherein the M-mode Doppler data representative of blood flow for the first and second carrier frequencies comprise:
reflected Doppler shift power data for a plurality of depths; and
spectrogram data for the plurality of depths.

46. The method of claim 44 wherein detecting the presence of the embolus based on power M-mode Doppler data representative of blood flow for the first carrier frequency comprises determining a ratio between power of the detected embolus and power from backscatter of blood.

47. The method of claim 44 wherein determining whether an embolic signature corresponding to the detected embolus is present based on power M-mode Doppler data representative of the blood flow for the second carrier frequency comprises determining a ratio between power of the detected embolus and power from noise.

48. The method of claim 47 wherein determining the ratio between power of the detected embolus and power from noise comprises calculating the power from power M-mode Doppler data representative of blood flow for which no emboli are detected.

49. The method of claim 47 wherein measuring the detected embolus peak power amplitude in a spectrogram and comparing the same with a background amplitude for a portion of the spectrogram having no embolus detected.

50. The method of claim 44 wherein the second carrier frequency is a second harmonic of the first carrier frequency.

51. An ultrasound transducer, comprising:
a first ultrasound transducer element to transmit an ultrasound signal at a transmission frequency and to detect reflected ultrasound signals; and
a second ultrasound transducer element to transmit an ultrasound signal at the transmission frequency and to detect reflected ultrasound signals,
a combination of the first and second ultrasound transducers having a first sample volume for a first reflected signal frequency in which the reflected ultrasound signals are detected and the second ultrasound transducer having a second sample volume for a second reflected signal frequency in which the reflected ultrasound signals are detected, the second sample volume substantially similar to the first sample volume.

52. The ultrasound transducer of claim 51 wherein the second and first ultrasound transducer elements are configured as a piston transducer and an annulus transducer, respectively.

53. The ultrasound transducer of claim 52 wherein the piston transducer and the annulus transducer form a cylindrical transducer having a front face that is flat.

54. The ultrasound transducer of claim 52 wherein the piston transducer and the annulus transducer form a cylindrical transducer having a concave front face with a radius of curvature.

55. The ultrasound transducer of claim 52 wherein the piston transducer has a first outside diameter and the annulus transducer has a second outside diameter that is twice the first outside diameter.

56. The ultrasound transducer of claim 51 wherein the second reflected signal frequency is a second harmonic of the first reflected signal frequency.

57. A method for generating an ultrasound signal and detecting reflected ultrasound signals, the method comprising:
detecting the reflected ultrasound signals for a first ultrasound field pattern at a first carrier frequency; and
detecting the reflected ultrasound signals for a second ultrasound field pattern at a second carrier frequency, the first and second ultrasound field patterns substantially similar.

58. The method of claim 57 wherein detecting the reflected ultrasound signals for the first ultrasound field pattern at the first carrier frequency comprises detecting the ultrasound signals from first and second ultrasound transducer elements and detecting the reflected ultrasound signals for the second ultrasound field pattern at a second carrier frequency comprises detecting the ultrasound signals from only the second ultrasound transducer element.

59. The method of claim 58 wherein the second and first ultrasound transducer elements are configured as a piston transducer and an annulus transducer, respectively.

60. The method of claim 59 wherein the piston transducer and the annulus transducer form a cylindrical transducer having a front face that is flat.

61. The method of claim 59 wherein the piston transducer and the annulus transducer form a cylindrical transducer having a concave front face with a radius of curvature.

62. The method of claim 58 wherein the piston transducer has a first outside diameter and the annulus transducer has a second outside diameter that is twice the first outside diameter.

63. The method of claim 57 wherein the second carrier frequency is a second harmonic of the first carrier frequency.
